(19) United States
(12) Patent Application Publication
(10) Pub. No.: US 2026/0080444 A1
(43) Pub. Date: Mar. 19, 2026

(54) METHOD FOR AUTOMATICALLY ASSOCIATING SURGICAL IMPLANT TYPE WITH SURGICAL MEDICAL BILLING CODES

(71) Applicant: Mitchell International, Inc., San Diego, CA (US)

(72) Inventors: Frederick William Stephens-Tiley, San Diego, CA (US); Teri Angelique Jones, San Diego, CA (US); Amelia Dae Vaughan, San Diego, CA (US)

(21) Appl. No.: 18/888,471

(22) Filed: Sep. 18, 2024

(51) Int. Cl. G06Q 30/04; G06Q 30/0283; G16H 10/60

(52) U.S. Cl. CPC G06Q 30/04; G06Q 30/0283; G16H 10/60

(57) ABSTRACT

Embodiments of the disclosure provide systems and methods for automated review of medical bills based on billing codes therein and surgical implant device types associated with those codes. Performing an automated review of medical bills can comprise maintaining a database of surgical implant device information and associated pricing information. When an electronic record for a medical bill is received, a determination can be made whether to review the electronic record for the medical bill based on information within the electronic record indicating use of a surgical implant device. In response to determining to review the electronic record for the medical bill, a price for the surgical implant device can be determined based on the electronic record for the medical bill and the database of surgical implant device information and associated pricing information. The determined price can be provided as a recommended payment amount for the surgical implant device.

### FIELD OF THE DISCLOSURE

Embodiments of the present disclosure relate generally to methods and systems for reviewing medical bills and more particularly to automated review of medical bills based on billing codes therein and surgical implant device types associated with those codes.

### BACKGROUND

Today, medical bills with surgical implants are reviewed using manual processes. In such cases, the invoice or billing information is reviewed relying on the knowledge and expertise of a human, reviewed and marked up, or edited if a price for the implant device should be changed. Such manual processes are time consuming, expensive, inconsistent, and error-prone. Hence, there is a need for improved methods and systems for reviewing medical bills with surgical implants.

### BRIEF SUMMARY

Embodiments of the disclosure provide systems and methods for automated review of medical bills with surgical implants based on billing codes therein and surgical implant device types associated with those codes. According to one embodiment, a method for performing an automated review of medical bills can comprise maintaining a database of surgical implant device information and associated pricing information, receiving an electronic record for a medical bill, and determining whether to review the electronic record for the medical bill based on information within the electronic record indicating use of a surgical implant device. For example, the information within the electronic record indicating use of a surgical implant device can comprise any one or more of information indicating a bill type, one or more line items, information indicating a body region, information indicating a type for the surgical implant device, and/or other information.

In response to determining to review the electronic record for the medical bill, a price can be determined for the surgical implant device based on the electronic record for the medical bill and the database of surgical implant device information and associated pricing information. Determining the price for the surgical implant device based on the electronic record for the medical bill and the database of surgical implant device information and associated pricing information can comprise reading, from the electronic record for the medical bill, information for the surgical implant device and surgical procedure, querying the database of surgical implant device information and associated pricing information for device type information associated with the surgical implant device, and querying the database of surgical implant device information and associated pricing information for device data for the surgical implant device. The device data can comprise historical pricing information based on the device type information associated with the surgical implant device. One or more predefined requirements can be applied to the device data. For example, the one or more predefined requirements can comprise a fee schedule and/or a definition of usual, customary, and reasonable pricing. A price for a line item of the electronic record for the medical bill can be generated based on the one or more predefined requirements applied to the device data and the determined price for the surgical implant device can be provided as a recommended payment amount for the surgical implant device.

According to another embodiment, a system can comprise a processor and a memory coupled with and readable by the processor. The processor can store therein a set of instructions which, when executed by the processor, causes the processor to maintain a database of surgical implant device information and associated pricing information, receive an electronic record for a medical bill, and determine whether to review the electronic record for the medical bill based on information within the electronic record indicating use of a surgical implant device. For example, the information within the electronic record indicating use of a surgical implant device can comprise any one or more of information indicating a bill type, one or more line items, information indicating a body region, information indicating a type for the surgical implant device, and/or other information.

In response to determining to review the electronic record for the medical bill, the instructions can further cause the processor to determine a price for the surgical implant device based on the electronic record for the medical bill and the database of surgical implant device information and associated pricing information. Determining the price for the surgical implant device based on the electronic record for the medical bill and the database of surgical implant device information and associated pricing information can comprise reading, from the electronic record for the medical bill, information for the surgical implant device and surgical procedure, querying the database of surgical implant device information and associated pricing information for device type information associated with the surgical implant device, and querying the database of surgical implant device information and associated pricing information for device data for the surgical implant device. The device data can comprise historical pricing information based on the device type information associated with the surgical implant device. The instructions can further cause the processor to apply one or more predefined requirements to the device data. For example, the one or more predefined requirements can comprise a fee schedule and/or a definition of usual, customary, and reasonable pricing. The instructions can further cause the processor to generate a price for a line item of the electronic record for the medical bill based on the one or more predefined requirements applied to the device data and provide the determined price for the surgical implant device as a recommended payment amount for the surgical implant device.

According to another embodiment, a non-transitory, computer-readable medium can comprise a set of instructions stored therein which, when executed by a processor, can cause the processor to maintain a database of surgical implant device information and associated pricing information, receive an electronic record for a medical bill, and determine whether to review the electronic record for the medical bill based on information within the electronic record indicating use of a surgical implant device. For example, the information within the electronic record indicating use of a surgical implant device can comprise any one or more of information indicating a bill type, one or more line items, information indicating a body region, information indicating a type for the surgical implant device, and/or other information.

In response to determining to review the electronic record for the medical bill, the instructions can further cause the processor to determine a price for the surgical implant device based on the electronic record for the medical bill and the database of surgical implant device information and associated pricing information. Determining the price for the surgical implant device based on the electronic record for the medical bill and the database of surgical implant device information and associated pricing information can comprise reading, from the electronic record for the medical bill, information for the surgical implant device and surgical procedure, querying the database of surgical implant device information and associated pricing information for device type information associated with the surgical implant device, and querying the database of surgical implant device information and associated pricing information for device data for the surgical implant device. The device data can comprise historical pricing information based on the device type information associated with the surgical implant device. The instructions can further cause the processor to apply one or more predefined requirements to the device data. For example, the one or more predefined requirements can comprise a fee schedule and/or a definition of usual, customary, and reasonable pricing. The instructions can further cause the processor to generate a price for a line item of the electronic record for the medical bill based on the one or more predefined requirements applied to the device data and provide the determined price for the surgical implant device as a recommended payment amount for the surgical implant device.

### BRIEF DESCRIPTION OF THE DRAWINGS

FIG. 1 is a block diagram illustrating elements of an exemplary computing environment in which embodiments of the present disclosure may be implemented.

FIG. 2 is a block diagram illustrating elements of an exemplary computing device in which embodiments of the present disclosure may be implemented.

FIG. 3 is a block diagram illustrating an environment in which automated medical bill review can be performed according to one embodiment of the present disclosure.

FIG. 4 is a flowchart illustrating an exemplary process for performing automated medical bill review according to one embodiment of the present disclosure.

FIG. 5 is a flowchart illustrating additional details of an exemplary process for determining pricing for a medical implant device according to one embodiment of the present disclosure.

In the appended figures, similar components and/or features may have the same reference label. Further, various components of the same type may be distinguished by following the reference label by a letter that distinguishes among the similar components. If only the first reference label is used in the specification, the description is applicable to any one of the similar components having the same first reference label irrespective of the second reference label.

### DETAILED DESCRIPTION

In the following description, for the purposes of explanation, numerous specific details are set forth in order to provide a thorough understanding of various embodiments disclosed herein. It will be apparent, however, to one skilled in the art that various embodiments of the present disclosure may be practiced without some of these specific details. The ensuing description provides exemplary embodiments only and is not intended to limit the scope or applicability of the disclosure. Furthermore, to avoid unnecessarily obscuring the present disclosure, the preceding description omits a number of known structures and devices. This omission is not to be construed as a limitation of the scopes of the claims. Rather, the ensuing description of the exemplary embodiments will provide those skilled in the art with an enabling description for implementing an exemplary embodiment. It should however be appreciated that the present disclosure may be practiced in a variety of ways beyond the specific detail set forth herein.

While the exemplary aspects, embodiments, and/or configurations illustrated herein show the various components of the system collocated, certain components of the system can be located remotely, at distant portions of a distributed network, such as a Local-Area Network (LAN) and/or Wide-Area Network (WAN) such as the Internet, or within a dedicated system. Thus, it should be appreciated, that the components of the system can be combined in to one or more devices or collocated on a particular node of a distributed network, such as an analog and/or digital telecommunications network, a packet-switch network, or a circuit-switched network. It will be appreciated from the following description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information.

Transmission media used as links, for example, can be any suitable carrier for electrical signals, including coaxial cables, copper wire and fiber optics, and may take the form of acoustic or light waves, such as those generated during radio-wave and infra-red data communications.

As used herein, the phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising,”“including,”and “having”can be used interchangeably.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

The term “computer-readable medium” as used herein refers to any tangible storage and/or transmission medium that participate in providing instructions to a processor for execution. Such a medium may take many forms, including but not limited to, non-volatile media, volatile media, and transmission media. Non-volatile media includes, for example, Non-Volatile Random-Access Memory (NVRAM), or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a Compact Disk Read-Only Memory (CD-ROM), any other optical medium, punch cards, paper tape, any other physical medium with patterns of holes, a Random-Access Memory (RAM), a Programmable Read-Only Memory (PROM), and Erasable Programable Read-Only Memory (EPROM), a Flash-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored.

A “computer readable signal” medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable medium may be transmitted using any appropriate medium, including but not limited to wireless, wireline, optical fiber cable, Radio Frequency (RF), etc., or any suitable combination of the foregoing.

The terms “determine,” “calculate,” and “compute,” and variations thereof, as used herein, are used interchangeably and include any type of methodology, process, mathematical operation or technique.

It shall be understood that the term “means” as used herein shall be given its broadest possible interpretation in accordance with 35 U.S.C., Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary of the disclosure, brief description of the drawings, detailed description, abstract, and claims themselves.

Aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Any combination of one or more computer readable medium(s) may be utilized. The computer readable medium may be a computer readable signal medium or a computer readable storage medium.

In yet another embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as Programmable Logic Device (PLD), Programmable Logic Array (PLA), Field Programmable Gate Array (FPGA), Programmable Array Logic (PAL), special purpose computer, any comparable means, or the like. In general, any device(s) or means capable of implementing the methodology illustrated herein can be used to implement the various aspects of this disclosure. Exemplary hardware that can be used for the disclosed embodiments, configurations, and aspects includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

Examples of the processors as described herein may include, but are not limited to, at least one of Qualcomm® Snapdragon® 800 and 801, Qualcomm® Snapdragon® 610 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K22 nm Haswell, Intel® Core® i5-3570K22 nm Ivy Bridge, the AMD® FX™ family of processors, AMD® FX-4300, FX-6300, and FX-8350 32 nm Vishera, AMD® Kaveri processors, Texas Instruments® Jacinto C6000™ automotive infotainment processors, Texas Instruments® OMAP™ automotive-grade mobile processors, ARM® Cortex™-M processors, ARM® Cortex-A and ARM926EJ-S™ processors, other industry-equivalent processors, and may perform computational functions using any known or future-developed standard, instruction set, libraries, and/or architecture.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or Very Large-Scale Integration (VLSI) design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or Common Gateway Interface (CGI) script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the aspects, embodiments, and/or configurations with reference to particular standards and protocols, the aspects, embodiments, and/or configurations are not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

Various additional details of embodiments of the present disclosure will be described below with reference to the figures. While the flowcharts will be discussed and illustrated in relation to a particular sequence of events, it should be appreciated that changes, additions, and omissions to this sequence can occur without materially affecting the operation of the disclosed embodiments, configuration, and aspects.

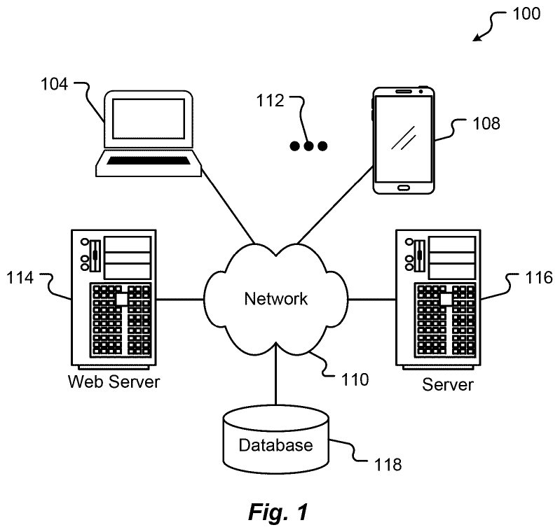

FIG. 1 is a block diagram illustrating elements of an exemplary computing environment in which embodiments of the present disclosure may be implemented. More specifically, this example illustrates a computing environment 100 that may function as the servers, user computers, or other systems provided and described herein. The environment 100 includes one or more user computers, or computing devices, such as a computing device 104, a communication device 108, and/or more 112. The computing devices 104, 108, 112 may include general purpose personal computers (including, merely by way of example, personal computers, and/or laptop computers running various versions of Microsoft Corp.'s Windows® and/or Apple Corp.'s Macintosh® operating systems) and/or workstation computers running any of a variety of commercially-available UNIX® or UNIX-like operating systems. These computing devices 104, 108, 112 may also have any of a variety of applications, including for example, database client and/or server applications, and web browser applications. Alternatively, the computing devices 104, 108, 112 may be any other electronic device, such as a thin-client computer, Internet-enabled mobile telephone, and/or personal digital assistant, capable of communicating via a network 110 and/or displaying and navigating web pages or other types of electronic documents. Although the exemplary computer environment 100 is shown with two computing devices, any number of user computers or computing devices may be supported.

Environment 100 further includes a network 110. The network 110 may can be any type of network familiar to those skilled in the art that can support data communications using any of a variety of commercially-available protocols, including without limitation Session Initiation Protocol (SIP), Transmission Control Protocol/Internet Protocol (TCP/IP), Systems Network Architecture (SNA), Internetwork Packet Exchange (IPX), AppleTalk, and the like. Merely by way of example, the network 110 maybe a Local Area Network (LAN), such as an Ethernet network, a Token-Ring network and/or the like; a wide-area network; a virtual network, including without limitation a Virtual Private Network (VPN); the Internet; an intranet; an extranet; a Public Switched Telephone Network (PSTN); an infra-red network; a wireless network (e.g., a network operating under any of the IEEE 802.9 suite of protocols, the Bluetooth® protocol known in the art, and/or any other wireless protocol); and/or any combination of these and/or other networks.

The system may also include one or more servers 114, 116. In this example, server 114 is shown as a web server and server 116 is shown as an application server. The web server 114, which may be used to process requests for web pages or other electronic documents from computing devices 104, 108, 112. The web server 114 can be running an operating system including any of those discussed above, as well as any commercially-available server operating systems. The web server 114 can also run a variety of server applications, including SIP servers, HyperText Transfer Protocol (secure) (HTTP(s)) servers, FTP servers, CGI servers, database servers, Java servers, and the like. In some instances, the web server 114 may publish operations available operations as one or more web services.

The environment 100 may also include one or more file and or/application servers 116, which can, in addition to an operating system, include one or more applications accessible by a client running on one or more of the computing devices 104, 108, 112. The server(s) 116 and/or 114 may be one or more general purpose computers capable of executing programs or scripts in response to the computing devices 104, 108, 112. As one example, the server 116, 114 may execute one or more web applications. The web application may be implemented as one or more scripts or programs written in any programming language, such as Java™, C, C#®, or C++, and/or any scripting language, such as Perl, Python, or Tool Command Language (TCL), as well as combinations of any programming/scripting languages. The application server(s) 116 may also include database servers, including without limitation those commercially available from Oracle®, Microsoft®, Sybase®, IBM® and the like, which can process requests from database clients running on a computing device 104, 108, 112.

The web pages created by the server 114 and/or 116 may be forwarded to a computing device 104, 108, 112 via a web (file) server 114, 116. Similarly, the web server 114 may be able to receive web page requests, web services invocations, and/or input data from a computing device 104, 108, 112 (e.g., a user computer, etc.) and can forward the web page requests and/or input data to the web (application) server 116. In further embodiments, the server 116 may function as a file server. Although for ease of description, FIG. 1 illustrates a separate web server 114 and file/application server 116, those skilled in the art will recognize that the functions described with respect to servers 114, 116 may be performed by a single server and/or a plurality of specialized servers, depending on implementation-specific needs and parameters. The computer systems 104, 108, 112, web (file) server 114 and/or web (application) server 116 may function as the system, devices, or components described herein.

The environment 100 may also include a database 118. The database 118 may reside in a variety of locations. By way of example, database 118 may reside on a storage medium local to (and/or resident in) one or more of the computers 104, 108, 112, 114, 116. Alternatively, it may be remote from any or all of the computers 104, 108, 112, 114, 116, and in communication (e.g., via the network 110) with one or more of these. The database 118 may reside in a Storage-Area Network (SAN) familiar to those skilled in the art. Similarly, any necessary files for performing the functions attributed to the computers 104, 108, 112, 114, 116 may be stored locally on the respective computer and/or remotely, as appropriate. The database 118 may be a relational database, such as Oracle 20i®, that is adapted to store, update, and retrieve data in response to Structured Query Language (SQL) formatted commands.

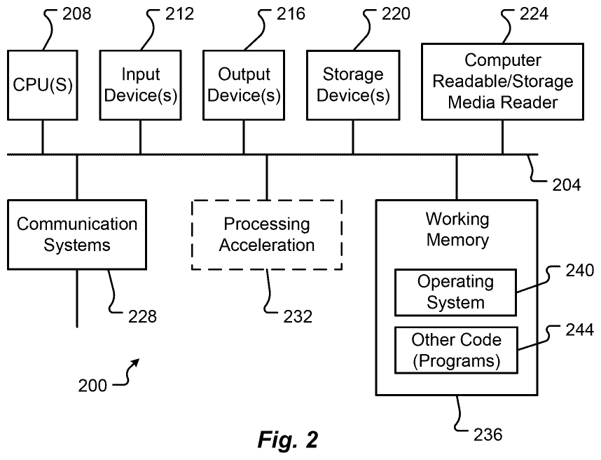

FIG. 2 is a block diagram illustrating elements of an exemplary computing device in which embodiments of the present disclosure may be implemented. More specifically, this example illustrates one embodiment of a computer system 200 upon which the servers, user computers, computing devices, or other systems or components described above may be deployed or executed. The computer system 200 is shown comprising hardware elements that may be electrically coupled via a bus 204. The hardware elements may include one or more Central Processing Units (CPUs) 208; one or more input devices 212 (e.g., a mouse, a keyboard, etc.); and one or more output devices 216 (e.g., a display device, a printer, etc.). The computer system 200 may also include one or more storage devices 220. By way of example, storage device(s) 220 may be disk drives, optical storage devices, solid-state storage devices such as a Random-Access Memory (RAM) and/or a Read-Only Memory (ROM), which can be programmable, flash-updateable and/or the like.

The computer system 200 may additionally include a computer-readable storage media reader 224; a communications system 228 (e.g., a modem, a network card (wireless or wired), an infra-red communication device, etc.); and working memory 236, which may include RAM and ROM devices as described above. The computer system 200 may also include a processing acceleration unit 232, which can include a Digital Signal Processor (DSP), a special-purpose processor, and/or the like.

The computer-readable storage media reader 224 can further be connected to a computer-readable storage medium, together (and, optionally, in combination with storage device(s) 220) comprehensively representing remote, local, fixed, and/or removable storage devices plus storage media for temporarily and/or more permanently containing computer-readable information. The communications system 228 may permit data to be exchanged with a network and/or any other computer described above with respect to the computer environments described herein. Moreover, as disclosed herein, the term “storage medium” may represent one or more devices for storing data, including ROM, RAM, magnetic RAM, core memory, magnetic disk storage mediums, optical storage mediums, flash memory devices and/or other machine-readable mediums for storing information.

The computer system 200 may also comprise software elements, shown as being currently located within a working memory 236, including an operating system 240 and/or other code 244. It should be appreciated that alternate embodiments of a computer system 200 may have numerous variations from that described above. For example, customized hardware might also be used and/or particular elements might be implemented in hardware, software (including portable software, such as applets), or both. Further, connection to other computing devices such as network input/output devices may be employed.

Examples of the processors 208 as described herein may include, but are not limited to, at least one of Qualcomm® Snapdragon® 800 and 801, Qualcomm® Snapdragon® 620 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22nm Haswell, Intel® Core® i5-3570K 22 nm Ivy Bridge, the AMD® FX™ family of processors, AMD® FX-4300, FX-6300, and FX-8350 32nm Vishera, AMD® Kaveri processors, Texas Instruments® Jacinto C6000™ automotive infotainment processors, Texas Instruments® OMAP™ automotive-grade mobile processors, ARM® Cortex™-M processors, ARM® Cortex-A and ARM926EJ-S™ processors, other industry-equivalent processors, and may perform computational functions using any known or future-developed standard, instruction set, libraries, and/or architecture.

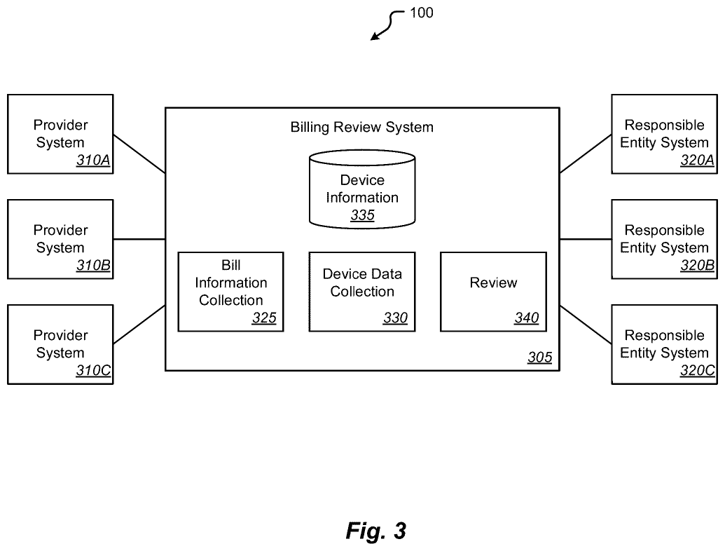

FIG. 3 is a block diagram illustrating an environment in which automated medical bill review can be performed according to one embodiment of the present disclosure. As illustrated in this example, the environment 300 can comprise a billing review system 305. The billing review system 305 can comprise any one or more servers and/or other computing devices as described above. The billing review system 305 can be coupled with a communication network (not shown here) such as described above. Through such a communication network, the billing review system 305 can receive invoices and other billing information from any number of provider systems 310A-310C such as may be used in hospitals, outpatient care facility, and/or other healthcare facilities. The billing review system 305 can review received invoices and/or billing information on behalf of one or more other entities such as insurance companies and/or other third-party payers responsible for or otherwise involved in payment of bills from the service providers. Based on this review, the billing review system 305 can provide over the communication network recommendations to any number of responsible entity systems 320A-320C for further review and/or processing.

According to one embodiment, the billing review system 305 can perform automated review of medical bills based on billing codes therein and surgical implant device types associated with those codes. More specifically, a bill information collection module 3255 and device data collection module 330 can maintain a database 335 of surgical implant device information and associated pricing information. The database 335 can comprise information including, but not limited to, information related a particular implant, implant type, implant category, a manufacturer, a manufacturer type, information related to billing codes, procedures, body parts, body part usage, and/or others. This information can be maintained over time and can comprise historical pricing data for a wide variety of implant devices. Sources for this data can include, but are not limited to, actual implant invoices with applicable data, externally acquired granular and aggregated implant invoice data, Bureau of Labor Statistics or other authority for inflationary adjustments applied to aged invoice data, other sources providing direction specific to payment for surgical implants.

At some point in time, an electronic record for a medical bill can be received. For example, the electronic record for the medical bill may be submitted by a provider system 310A of a medical service provider such as a hospital or outpatient care facility. The electronic record can comprise information such as codes, for example, indicating use of an implant. For example, the information within the electronic record indicating use of a surgical implant device can comprise any one or more of information indicating a bill type, one or more codes such as revenue codes, procedure codes, diagnosis codes, etc., information indicating a body region or sub-region upon which a procedure was performed, information indicating a type for the surgical implant device, and/or other information.

More specifically, the information in the electronic record can indicate a bill type. The bill types can be used to determine whether an implant associated with a surgical procedure can be billed. Such bill types can include Inpatient Hospital (IPH), Outpatient Hospital (OPH), and Ambulatory Surgical Center (ASC). The information in the electronic record can include various codes such as revenue codes, procedure codes, and/or diagnosis codes. Revenue codes are similar to a cost center in accounting terms and can include notes on a medical bill that an implant was used in the surgical procedure. Such codes can include “272, Medical/Surgical Supplies and Devices—Sterile,” “278, Medical/Surgical Supplies and Devices—Other Implants,” or “279, Medical/Surgical Supplies and Devices—Other Supplies/Devices.” Procedure codes are five digit code that represent the actual medical procedure performed which, if applicable, is associated with implant types. These codes determine the body region, and or sub-region that was repaired. Such codes can include “ICD-10 Procedure—IPH bills” or “CPT—OPH and ASC bills.” Diagnosis codes are codes that describe the nature of the injury/ailment the patient is experiencing and potentially any surgical procedure performed on them to remedy the injury/ailment. Such codes can include a Diagnosis Related Group (DRG) such as “ICD-10.” The information in the electronic record can also include notes on body regions and/or body sub-regions, i.e., parts of the human body such as arm or leg that is used to associate the surgical procedure performed to the type of implant used. The type of implants that are used in surgical procedures can include, but are not limited to, plates, screws, rods, wire, mesh, spacers, allograft, cages, matrix, putty, pins, fibrin, stimulator leads, pegs, stents, anchors, harpoons, prosthesis and others, any of which may be indicated in the information in the electronic record.

A review module 340 can make a determination as to whether to review the electronic record for the medical bill based on the information within the electronic record indicating use of the surgical implant device, i.e., the codes and/or other information described above. In response to determining to review the electronic record for the medical bill, a price can be determined for the surgical implant device based on the electronic record for the medical bill and the database of surgical implant device information and associated pricing information.

Determining the price for the surgical implant device based on the electronic record for the medical bill and the database of surgical implant device information and associated pricing information can comprise reading, from the electronic record for the medical bill, information for the surgical implant device and surgical procedure. As noted above, this information can include, but is not limited to, information identifying the implant, the procedure performed, etc. The database of surgical implant device information and associated pricing information can then be queried for device type information associated with the surgical implant device. The database of surgical implant device information and associated pricing information can also be queried for device data for the surgical implant device. The device data can comprise historical pricing information based on the device type information associated with the surgical implant device.

One or more predefined requirements can be applied to the device data. For example, the one or more predefined requirements can comprise a fee schedule and/or a definition of Usual, Customary, and Reasonable (UCR) pricing. For example, for workers compensation and third-party injury, many states have a mandated fee schedule that determines the reimbursement to medical providers for the specific services rendered to these patient populations. Some state statutes, and health plans may refer to UCR language, either statutory or in plan documents, that provides direction regarding how payments for surgical implants are reimbursed.

A price for a line item of the electronic record for the medical bill can then be generated based on the one or more predefined requirements applied to the device data. Once determined, the price for the surgical implant device can be provided as a recommended payment amount for the surgical implant device. For example, this can be provided through a graphical user interface, a report, an invoice, etc. to a system 320A of an entity responsible for or involved in the payment of the bill such as an insurance company or other third-party payer.

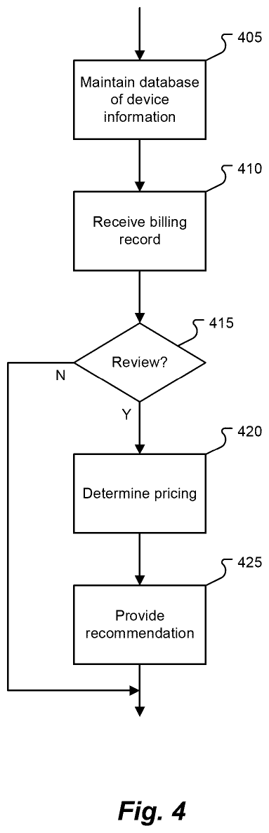

FIG. 4 is a flowchart illustrating an exemplary process for performing automated medical bill review according to one embodiment of the present disclosure. As illustrated in this example, performing an automated review of medical bills can comprise maintaining 405 a database of surgical implant device information and associated pricing information. As noted above, the database can comprise information including, but not limited to, information related a particular implant, implant type, implant category, a manufacturer, a manufacturer type, information related to billing codes, procedures, body parts, body part usage, and/or others. This information can be maintained over time and can comprise historical pricing data for a wide variety of implant devices.

At some point in time, an electronic record for a medical bill can be received 410. For example, the electronic record for the medical bill may be submitted by a system of medical service provider such as a hospital or outpatient care facility. As noted above, the electronic record can comprise information such as codes, for example, indicating use of an implant. For example, the information within the electronic record indicating use of a surgical implant device can comprise any one or more of information indicating a bill type, one or more codes such as revenue codes, procedure codes, diagnosis codes, etc., information indicating a body region or sub-region upon which a procedure was performed, information indicating a type for the surgical implant device, and/or other information.

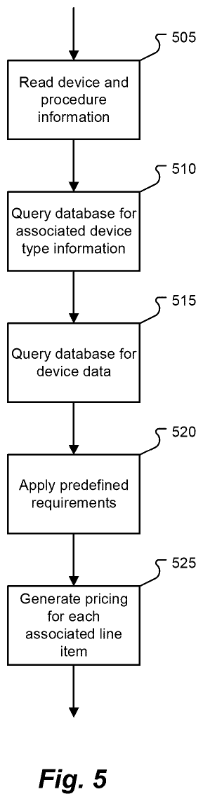

A determination 415 can be made as to whether to review the electronic record for the medical bill based on the information within the electronic record indicating use of the surgical implant device. In response to determining 415 to review the electronic record for the medical bill, a price can be determined 420 for the surgical implant device. Generally speaking, the price can be determined 420 based on the electronic record for the medical bill and the database of surgical implant device information and associated pricing information. Additional details of an exemplary process for determining 420 the price will be described below with reference to FIG. 5. Once determined 420, the price for the surgical implant device can be provided 425 as a recommended payment amount for the surgical implant device. For example, this can be provided through a graphical user interface, a report, an invoice, etc. to an entity responsible for or involved in the payment of the bill such as an insurance company.

FIG. 5 is a flowchart illustrating additional details of an exemplary process for determining pricing for a medical implant device according to one embodiment of the present disclosure. As illustrated in this example, determining the price for the surgical implant device based on the electronic record for the medical bill and the database of surgical implant device information and associated pricing information can comprise reading 505, from the electronic record for the medical bill, information for the surgical implant device and surgical procedure. As noted above, this information can include, but is not limited to, information identifying the implant, the procedure performed, etc. The database of surgical implant device information and associated pricing information can then be queried 510 for device type information associated with the surgical implant device. The database of surgical implant device information and associated pricing information can also be queried 515 for device data for the surgical implant device. The device data can comprise historical pricing information based on the device type information associated with the surgical implant device. One or more predefined requirements can be applied 520 to the device data. For example, the one or more predefined requirements can comprise a fee schedule and/or a definition of usual, customary, and reasonable pricing. A price for a line item of the electronic record for the medical bill can then be generated 525 based on the one or more predefined requirements applied to the device data.

The present disclosure, in various aspects, embodiments, and/or configurations, includes components, methods, processes, systems, and/or apparatus substantially as depicted and described herein, including various aspects, embodiments, configurations embodiments, sub-combinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and/or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. A method for performing an automated review of medical bills with surgical implants, the method comprising:
maintaining, by a processor of a billing review system, a database of surgical implant device information and associated pricing information, the device data comprising historical pricing information based on the device type information associated with the surgical implant device;
receiving, by the processor of the billing review system, an electronic record for a medical bill;
determining, by the processor of the billing review system, whether to review the electronic record for the medical bill based on information within the electronic record indicating use of a surgical implant device;
in response to determining to review the electronic record for the medical bill, determining, by the processor of the billing review system, a price for the surgical implant device based on the electronic record for the medical bill and the database of surgical implant device information and associated pricing information; and
providing, by the processor of the billing review system, the determined price for the surgical implant device as a recommended payment amount for the surgical implant device.

2. The method of claim 1, wherein the information within the electronic record indicating use of a surgical implant device comprises information indicating a bill type.

3. The method of claim 1, wherein the information within the electronic record indicating use of a surgical implant device comprises one or more line items.

4. The method of claim 1, wherein the information within the electronic record indicating use of a surgical implant device comprises information indicating a body region.

5. The method of claim 1, wherein the information within the electronic record indicating use of a surgical implant device comprises information indicating a type for the surgical implant device.

6. The method of claim 1, wherein determining the price for the surgical implant device based on the electronic record for the medical bill and the database of surgical implant device information and associated pricing information comprises:
reading, from the electronic record for the medical bill, information for the surgical implant device and surgical procedure;
querying the database of surgical implant device information and associated pricing information for device type information associated with the surgical implant device;
querying the database of surgical implant device information and associated pricing information for device data for the surgical implant device,
applying one or more predefined requirements to the device data; and
generating a price for a line item of the electronic record for the medical bill based on the one or more predefined requirements applied to the device data.

7. The method of claim 6, wherein the one or more predefined requirements comprise a fee schedule.

8. The method of claim 6, wherein the one or more predefined requirements comprise a definition of usual, customary, and reasonable pricing.

9. A system comprising:
a processor; and
a memory coupled with and readable by the processor and storing therein a set of instructions which, when executed by the processor, causes the processor to:
maintain a database of surgical implant device information and associated pricing information, the device data comprising historical pricing information based on the device type information associated with the surgical implant device;
receive an electronic record for a medical bill;
determine whether to review the electronic record for the medical bill based on information within the electronic record indicating use of a surgical implant device;
in response to determining to review the electronic record for the medical bill, determine a price for the surgical implant device based on the electronic record for the medical bill and the database of surgical implant device information and associated pricing information; and
provide the determined price for the surgical implant device as a recommended payment amount for the surgical implant device.

10. The system of claim 9, wherein the information within the electronic record indicating use of a surgical implant device comprises information indicating a bill type.

11. The system of claim 9, wherein the information within the electronic record indicating use of a surgical implant device comprises one or more line items.

12. The system of claim 9, wherein the information within the electronic record indicating use of a surgical implant device comprises information indicating a body region.

13. The system of claim 9, wherein the information within the electronic record indicating use of a surgical implant device comprises information indicating a type for the surgical implant device.

14. The system of claim 9, wherein determining the price for the surgical implant device based on the electronic record for the medical bill and the database of surgical implant device information and associated pricing information comprises:
reading, from the electronic record for the medical bill, information for the surgical implant device and surgical procedure;
querying the database of surgical implant device information and associated pricing information for device type information associated with the surgical implant device;
querying the database of surgical implant device information and associated pricing information for device data for the surgical implant device,
applying one or more predefined requirements to the device data; and
generating a price for a line item of the electronic record for the medical bill based on the one or more predefined requirements applied to the device data.

15. The system of claim 14, wherein the one or more predefined requirements comprise a fee schedule.

16. The system of claim 14, wherein the one or more predefined requirements comprise a definition of usual, customary, and reasonable pricing.

17. A non-transitory, computer-readable medium comprising a set of instructions stored therein which, when executed by a processor, causes the processor to:
maintain a database of surgical implant device information and associated pricing information, the device data comprising historical pricing information based on the device type information associated with the surgical implant device;
receive an electronic record for a medical bill;
determine whether to review the electronic record for the medical bill based on information within the electronic record indicating use of a surgical implant device;
in response to determining to review the electronic record for the medical bill, determine a price for the surgical implant device based on the electronic record for the medical bill and the database of surgical implant device information and associated pricing information; and
provide the determined price for the surgical implant device as a recommended payment amount for the surgical implant device.

18. The non-transitory, computer-readable medium of claim 17, wherein the information within the electronic record indicating use of a surgical implant device comprises one or more of information indicating a bill type, one or more codes, information indicating a body region, or information indicating a type for the surgical implant device.

19. The non-transitory, computer-readable medium of claim 17, wherein determining the price for the surgical implant device based on the electronic record for the medical bill and the database of surgical implant device information and associated pricing information comprises:
reading, from the electronic record for the medical bill, information for the surgical implant device and surgical procedure;
querying the database of surgical implant device information and associated pricing information for device type information associated with the surgical implant device;
querying the database of surgical implant device information and associated pricing information for device data for the surgical implant device,
applying one or more predefined requirements to the device data; and
generating a price for a line item of the electronic record for the medical bill based on the one or more predefined requirements applied to the device data.

20. The non-transitory, computer-readable medium of claim 19, wherein the one or more predefined requirements comprise one or more of a fee schedule or a definition of usual, customary, and reasonable pricing.
